(19) United States
(12) Patent Application Publication
(10) Pub. No.: US 2014/0271866 A1
(43) Pub. Date: Sep. 18, 2014

(54) TRANSDERMAL DRUG DELIVERY SYSTEM CONTAINING RIVASTIGMINE

(71) Applicant: NAL PHARMACEUTICALS, LTD., CAUSEWAY BAY (HK)

(72) Inventor: Je Phil Ryoo, Princeton, NJ (US)

(73) Assignee: NAL PHARMACEUTICALS, LTD., CAUSEWAY BAY (HK)

(21) Appl. No.: 14/211,373

(22) Filed: Mar. 14, 2014

Related U.S. Application Data
(60) Provisional application No. 61/799,015, filed on Mar. 15, 2013.

(51) Int. Cl. A61K 47/32; A61K 9/70; A61K 31/27

(52) U.S. Cl. CPC A61K 47/32; A61K 31/27; A61K 9/7023
USPC 424/487; 514/490; 604/307

(57) ABSTRACT

The present invention provides a transdermal drug delivery system comprising rivastigmine or its pharmaceutically acceptable salt and method of making the same.

### CROSS-REFERENCE TO RELATED APPLICATION

This application claims the benefit of priority from U.S. Provisional Patent Application Ser. No. 61/799,015 filed Mar. 15, 2013, the contents of which are incorporated herein by reference.

### FIELD OF THE INVENTION

The present invention relates to a transdermal drug delivery system comprising rivastigmine or its pharmaceutically acceptable salt and method of making the same.

### BACKGROUND OF THE INVENTION

Dementia is a clinical syndrome characterized by deficits in multiple areas of cognition that cannot be explained by normal aging, a noticeable decline in function, and an absence of delirium. Alzheimer's disease and Parkinson's disease are forms of dementia that gradually gets worse over time. It affects memory, thinking, and behavior.

In the brain, neurons connect and communicate at synapses, where tiny bursts of chemicals called neurotransmitters carry information from one cell to another. Alzheimer's disrupts this process, and eventually destroys synapses and kills neurons, damaging the brain's communication network.

Alzheimer's disease damages or destroys cells that produce and use acetylcholine, thereby reducing the amount available to carry messages. A cholinesterase inhibitor slows the breakdown of acetylcholine by blocking the activity of acetylcholinesterase. By maintaining acetylcholine levels, the drug may help compensate for the loss of functioning brain cells.

Current drugs help mask the symptoms of Alzheimer's or Parkinson's, but do not treat the underlying disease. The FDA has approved the following cholinesterase inhibitors to treat the symptoms of Alzheimer's disease and Parkinson's disease, which work by slowing down the disease activity that breaks down a key neurotransmitter: Donepezil (Aricept), galantamine (Nivalin, Razadyne, Razadyne ER, Reminyl, Lycoremine), rivastigmine (Exelon). Donepezil is approved to treat all stages of Alzheimer's, while Rivastigmine and Galantamine are approved to treat mild to moderate Alzheimer's.

Among the acetylcholinesterase inhibitors, rivastigmine has been available in capsule and liquid formulations since 1997. In 2006 it became the first product approved globally for the treatment of mild to moderate dementia associated with Parkinson's disease, and in 2007 the rivastigmine transdermal patch became the first patch treatment for dementia. In patients with either type of dementia (i.e. Alzheimer's and Parkinson's patients), rivastigmine has been known to provide meaningful symptomatic effects that may allow patients to remain independent and ‘be themselves’ for longer. Rivastigmine is believed to work by blocking the activity of another enzyme involved in the breaking down of acetylcholine.

Rivastigmine transdermal patch is sold under the trade name Exelon, which is a double layer composition, where the first layer comprises the rivastigmine in polyacrylate and methacrylate matrix with an antioxidant such as alpha-tocopherol, and where the second layer comprises a silicon base adhesive. However, the Exelon patch can cause gastrointestinal adverse reactions, including significant nausea, vomiting, loss of appetite and weight loss. Other side effects include skin irritations.

There is still a great need for simple and effective ways of manufacturing a transdermal drug delivery system with effective amounts of drugs being delivered during the treatment for mild to moderate dementia, such as Alzheimer's and Parkinson's disease. The present invention addresses this need.

### SUMMARY OF INVENTION

The present invention provides a transdermal drug delivery system comprising rivastigmine or its pharmaceutically acceptable salt. The present invention not only provides high skin penetration rate but also continuous maintenance of a therapeutically effective blood concentration for at least 24 hours. Additionally, the present invention provides a transdermal drug delivery system which can inhibit recrystallization of rivastigmine while maintaining skin penetration rate intact, even during long-term storage. Further, the present invention maintains stability and adhesion strength without requiring any antioxidants and additional adhesive layers.

Thus, the present invention provides a rivastigmine-containing transdermal drug delivery system having high skin penetration rate continuously up to or for more than 24 hours with excellent stability.

### BRIEF DESCRIPTION OF THE DRAWINGS

FIG. 1 shows the stability study of the formulations RN-3, RN-4, RN-5, RN-6, RN-7 and RN-8 found in Table 1 at 60° C. and at 40° C.;

FIG. 2 shows the stability of the formulations RN-11, RN-14 and RN-17 found in Table 3 at 60° C. and at 40° C. compared to the Exelon patch;

FIG. 3 shows the stability of the formulations RN-18, RN-19 and RN-20 found in Table 4 at 60° C. and at 40° C. compared to the Exelon patch;

FIG. 4 shows comparative in vitro human skin permeation results of RN-18 and the Exelon patch found in Table 6;

FIG. 5 shows comparative data of formulation RN-18 and the Exelon patch; and

FIG. 6 shows comparative formula and Stability study of RN-18 and the Exelon patch.

### DETAILED DESCRIPTION OF INVENTION

In one aspect of the present invention, there is provided a transdermal drug delivery system comprising a drug-containing matrix layer comprising rivastigmine or its pharmaceutically acceptable salt and an acrylic-hydrocarbon hybrid polymer adhesive.

In an embodiment according to the present invention, the transdermal drug delivery system may comprise a backing layer, a drug-containing matrix layer and a release layer.

As used herein, the term “acrylic-hydrocarbon hybrid polymer” adhesives refers to an acrylic polymer grafted with a hydrocarbon macromer including.

The acrylic-hydrocarbon hybrid polymer according to the invention may be an acrylic polymer comprising a C₄₋₁₈ alkyl acrylate monomer grafted with a hydrocarbon macromer having a glass transition temperature of no more than −30° C. The acrylic-hydrocarbon hybrid polymer adhesive may be present in an amount ranging from about 60 to about 95% by weight based on the total weight of the drug-containing matrix layer, alternatively may be present from about 70 to about 90%, or from about 75 to about 85%. The acrylic-hydrocarbon hybrid polymer adhesive of the invention may be one or more selected from commercially available acrylic-hydrocarbon hybrid polymers, i.e. Duro-Tak™ 87-502B (National Starch) and Duro-Tak™ 87-504B (National Starch), Duro-Tak™ 87-502A (National Starch), Duro-Tak™ 87-503A (National Starch) and Duro-Tak™ 87-504A (National Starch).

In the transdermal drug delivery system according to the present invention, the acrylic-hydrocarbon hybrid polymer is used as an adhesive and the acrylic-hydrocarbon hybrid polymer adhesive forms a matrix in the drug-containing matrix layer. In other words, rivastigmine or its pharmaceutically acceptable salt is homogenously dispersed in the acrylic-hydrocarbon hybrid polymer adhesive thereby forming the drug-containing matrix layer.

Some examples of the acrylic-hydrocarbon hybrid adhesives used can be include the three different types as provided in Table A (below), which can be classified according to the presence of a cross-liking agent and a tackifier. Also, it can be distinguished by two groups of solvent system (Table B). The compositions of two solvent systems [Group A (502A, 503A and 504B) & Group B (502B and 504B)] are described in Table B. During the formulation development, the solid part of adhesive is dissolved in the solvents, which the drug substance and other excipients can be dissolved in.

TABLE A
Types of Hybrid Pressure Sensitive Adhesive (PSA)

| | | Functional | Cross linker |
|---|---|---|---|
| PSA | Chemical composition | group | added |
| 87-502A | Acrylic-hydrocarbon hybrid | -OH | X |
| 87-502B | | | |
| 87-503A | Acrylic-hydrocarbon hybrid | | O |
| 87-504A | Acrylic-hydrocarbon hybrid | | O |
| 87-504B | tackifier | | |

TABLE B
Solvent System of Hybrid PSA

| PSA | SOLVENT (%) |
|---|---|
| 87-502A, 87-503A, 87-504A | Ethyl acetate: 45 |
| | n-heptane: 31 |
| | n-hexane: 24 |
| 87-502B | Ethyl acetate: 30-60 |
| | n-heptane: 10-30 |
| 87-504B | Ethyl acetate: 30-60 |
| | n-heptane: 10-30 |
| | Acetylacetone: 0.1-1 |

Therefore, even though the chemical structure of an adhesive may be known, the formulation for developing a transdermal patch should be modified significantly according to the solvent compositions. Since their physical properties and the compatibility of adhesives to drug substance were changed, their formulation development of patch should be approached with totally different methods to maintain the better stability of the final formula.

It has been surprisingly found that the matrix formed from the acrylic-hydrocarbon hybrid polymer having low glass transition temperature according to the invention can improve the flexibility of polymer chains increases the diffusion rate of the active ingredient, i.e. rivastigmine or its pharmaceutically acceptable salt. Accordingly, the acrylic-hydrocarbon hybrid polymer provides higher skin penetration rate and excellent adhesive force, when compared to using only acrylic adhesives having no functional groups (e.g., Duro-Tak™ 87-4098, Duro-Tak™ 87-900A, Duro-Tak™ 87-9301, etc.) or other types of acrylic adhesives having hydroxyl or carboxyl functional group (e.g., Duro-Tak™ 87-2516, Duro-Tak™ 87-2510, Duro-Tak™ 87-2525, Duro-Tak™ 87-2596, Duro-Tak™ 87-2825, Duro-Tak™ 87-2502, Duro-Tak™ 87-2979, Duro-Tak™ 87-2074, Duro-Tak™ 87-2353 etc.).

The acrylic-hydrocarbon hybrid polymer adhesive may be used in an amount sufficient to form a matrix layer, for example, in an amount ranging from about 60% to about 90% by weight based on the total weight of the drug-containing matrix layer, alternatively may be present from about 70 to about 90%, or from about 75 to about 85%.

In the transdermal drug delivery system according to the present invention, rivastigmine or its pharmaceutically acceptable salt may be present in an amount ranging from about 5 to about 40% based on the total weight of the drug-containing matrix layer. In an embodiment rivastigmine or its pharmaceutically acceptable salt may be present in an amount ranging from about 7 to about 30%, or from about 10 to about 20%.

If the amount of rivastigmine or its pharmaceutically acceptable salt is more than 40% by weight, drug crystals may be formed in the transdermal drug delivery system, which results in reducing adhesive force or lowering absorption rate of the drug.

Additionally, the transdermal drug delivery system according to the present invention may comprise an absorption enhancer used in the field of transdermal drug delivery system. The absorption enhancer may be present in an amount ranging from about 1% to about 20% by weight, preferably from about 5% to about 15% by weight based on the total weight of the drug-containing matrix layer. If the amount of the absorption enhancer is more than 20% by weight, adhesive force may be reduced or cold flow may occur due to the weakened cohesive force.

The transdermal drug delivery system according to the present invention may further comprise one or more absorption enhancers selected from among terpenes, surfactants, polyoxyethylene alkyl ethers, fatty alcohols, sugar esters, glycerols, alkyl 2-ethyl hexanates and diethoxyethyl succinates. The absorption enhancers may be present in an amount ranging from about 1% to about 20% by weight based on the total weight of the drug-containing matrix layer. The absorption enhancer may be one or more selected from among polyethylene glycol palm kernel glyceride, polyoxyethylene lauryl ether, polyglyceryl-3-oleate, lauryl alcohol and oleyl alcohol.

Examples of terpenes include cineole, limonene, etc.

Examples of surfactants include isopropyl myristate, isopropyl palmitate, 2-(2-ethoxyethoxy) ethanol, oleic acid oleyl ester, caprylocaproyl macrogolglyceride, oleoyl macrogolglyceride, diisopropyl dirrerate, diisopropyl adipate, hexyl laurate, polysorbate, sorbitan oleate, etc.

Examples of polyoxyethylene alkyl ethers include polyethylene glycol palm kernel glyceride, 2-ethyl hexyl hydroxystearate, polyoxyethylene lauryl ether, polyoxyethylene cetyl ether, etc.

Examples of fatty alcohols include polyglyceryl-3 oleate, polyethylene glycol almond glyceride, lauryl alcohol, oleyl alcohol, etc.

Examples of sugar esters include sucrose stearate, sucrose palmitate, sucrose laurate, sucrose behenate, sucrose oleate, sucrose erucate, etc.

Examples of alkyl 2-ethyl hexanates include 2-ethylhexanonate, cetyl 2-ethylhexanonate, stearyl 2-ethylhexanonate, etc.

Among the above mentioned absorption enhancers, polyoxyethylene alkyl ethers and/or fatty alcohols may be preferably used. More preferable, the absorption enhancer may be one or more selected from among polyethylene glycol palm kernel glyceride (e.g. Crovol™ A40), polyoxyethylene lauryl ether (e.g. Brij™ 30, Brij™ 52, etc), polyglyceryl-3 oleate (e.g. Plurol oleique™ cc497), lauryl alcohol, and oleyl alcohol. Most preferably, polyoxyethylene lauryl ether (e.g. Brij™ 30) may be used as an absorption enhancer.

Some of the advantages conferred by the present invention include, for example, increased diffusion rate of rivastigmine from the matrix layer, high skin penetration rate, continuous maintenance of a therapeutically effective blood concentration for at least 24 hours, inhibition of recrystallization of rivastigmine, maintenance of skin penetration rate even during long-term storage, improvement of drug compliance of patients. Further advantages include, for example, easy manufacture as it is a single layer, less dosing required due to high permeation (e.g., less drug content needed to deliver such as 30% less than Exelon's patch). Moreover, the size of the patch according to the invention can range from about 2.5 cm² to about 20 cm², e.g. 3.5, 5, 7, 10 10.5, or 15 cm², depending on the area to be applied.

Rivastigmine, which is marketed under the trade name Exelon, is a reversible acetylcholinesterase (ACE) inhibitor which treats mild to moderate dementia of the Alzheimer's type or that associated with Parkinson's disease. Rivastigmine increased cortical acetylcholine thereby improving transmission of electrical signals across certain areas of the brain. However, it has a short half-life, i.e. about 1.5 hours.

The present invention provides for a 1-Day patch which prolongs the duration of the drug over a period of 24 hours. With the convenient once-a-day therapy, it encourages and improves patient compliance. Not only is it convenient to the patient for compliance but it lessens the burden of the monitoring caretaker.

The present inventions provides for a more consistent drug plasma profile versus that of the oral dosage form, which has a short half-life of about 1.5 hour.

Further, the present invention allows for topical application which avoids gastrointestinal irritations, especially for elderly patients. With the present invention, topical application bypasses the first-pass liver metabolism side effects.

The transdermal drug delivery system of the present invention may be prepared by forming the drug-containing matrix layer on a release layer and then forming a backing layer thereon. For the release layer, conventional release liners or their laminates used in the field of transdermal drug delivery system may be used. For example, a film, a paper or laminates thereof which are made of polyethylene, polyester, polyvinyl chloride, polyvinylidene chloride, etc. coated with silicon resin or fluoride resin.

Additionally, drug non-absorbable and flexible materials conventionally sued in the field of transdermal drug delivery system may be used as the backing layer (also referred to as “backing membrane”). For example, they may be polyolefin, polyether, multi-layer ethylene vinyl acetate film, polyester, polyurethane, etc. The transdermal drug delivery system of the present invention may be prepared, for example, by dissolving rivastigmine or its pharmaceutically acceptable slat and an acrylic-hydrocarbon hybrid polymer adhesive, optionally along with an absorption enhancer in an appropriate solvent (e.g., ethyl acetate), casting the resulting solution on a release liner coated with silicon followed by drying the mixture and then laminating a backing layer.

Some preparation examples of the present invention are provided below. These examples are illustrative, but not limiting the scope of the present invention. Reasonable variations can be made herein without departing from the scope of the present invention.

### EXAMPLES

### General Method of Making the Transdermal Patch

Mixture A: to a solution of rivastigmine base in ethyl acetate, an enhancer was added.

Mixture B: to Mixture A, a hybrid adhesive (Henkel, USA) was further added and stirred thoroughly until a uniform Mixture C was obtained.

Drug-Adhesive Matrix Layer: Mixture C was cast on release liner (3M Scotchpak 1022) coated with silicone and all solvents were removed by evaporation at room temperature for 20 minutes and subsequently oven-dried at 80° C. for 15 minutes.

A backing film which consists of a translucent flexible polyethylene film (3M Cotran 9735) was also laminated on the drug-adhesive matrix layer. The obtained laminated sheet was cut into a size of 10 cm² by a die-cutting machine. The dug loading was adjusted to 18 mg/cm² per unit area. The complete patch was immediately pouched with PET/AL/PAN packaging material.

| | |
|---|---|
| rivastigmine base | 10~30% |
| Adhesive (acrylic-hydrocarbon hybrid | 65~85% |
| polymer) | |
| Enhancer* (Lauroglycol 90 or Plurol | 5% |
| oleique CC 497 or Labrasol | |
| Total | 100% |

*Lauroglycol 90: Propylene glycol monolaurate, Plurol oleique CC 497: Polyglyceryl-3 dioleate, Labrasol: Caprylocaproyl polyoxyl-8 glycerides

### General Testing Method

1) Crystallization

The crystals in the patch during the preliminary stability study period were observed by naked eye or a microscope at different storage conditions.

2) Adhesion

The patch's peel adhesion value was measured using Instron or a texture analyzer, and then classified as being sufficient, slightly sufficient or insufficient.

3) Bleeding

The bleeding of drug from the patch was observed by naked eye. The bleeding was also checked by wiping a clean tissue on the surface of the patch.

TABLE 1
Examples of Patch formulations RN-3 to RN-8 containing Rivastigmine

| | Adhesive | Adhesive | Adhesive | | Lauroglycol | Plurol oleique | | |
|---|---|---|---|---|---|---|---|---|
| Patch | (17408) | (M) | (S) | Rivastigmine* | 90 | CC 497 | Labrasol | Thickness |
| RN-3 | 70 | | | 25 | | 5 | | 72 μm |
| RN-4 | 65 | | | 30 | | 5 | | 60 μm |
| RN-5 | 65 | | | 30 | 5 | | | 60 μm |
| RN-6 | 70 | | | 25 | 5 | | | 72 μm |
| RN-7 | 65 | | | 30 | | | 5 | 60 μm |
| RN-8 | 70 | | | 25 | | | 5 | 72 μm |

FIG. 1 shows the stability of drug content of the above formulations in different conditions.

All patch formulations listed in the above Table 1 tested for stability. The patches were packaged in aluminum pouch stored in stability chambers with different storage conditions (60° C./75% RH, 40° C./75% RH, 25° C./60% RH). At the designated time period, the patch formulations were taken and tested on several attributes (crystallization, adhesion and bleeding).

TABLE 2
Stability Study

| | | | 60° C. | | 40° C. | | | 25° C. | | |
|---|---|---|---|---|---|---|---|---|---|---|
| | Patch | Initial | 1 M | 2 M | 1 M | 2 M | 3 M | 1 M | 2 M | 3 M |
| Crystallization | RN-3 | None | None | None | None | None | None | None | None | None |
| | RN-4 | None | None | None | None | None | None | None | None | None |
| | RN-5 | None | None | None | None | None | None | None | None | None |
| | RN-6 | None | None | None | None | None | None | None | None | None |
| | RN-7 | None | None | None | None | None | None | None | None | None |
| | RN-8 | None | None | None | None | None | None | None | None | None |
| Adhesion* | RN-3 | ∘ | ∘ | ∘ | ∘ | ∘ | ∘ | ∘ | ∘ | ∘ |
| | RN-4 | ∘ | ∘ | ∘ | ∘ | ∘ | ∘ | ∘ | ∘ | ∘ |
| | RN-5 | ∘ | ∘ | ∘ | ∘ | ∘ | ∘ | ∘ | ∘ | ∘ |
| | RN-6 | ∘ | ∘ | ∘ | ∘ | ∘ | ∘ | ∘ | ∘ | ∘ |
| | RN-7 | ∘ | ∘ | ∘ | ∘ | ∘ | ∘ | ∘ | ∘ | ∘ |
| | RN-8 | ∘ | ∘ | ∘ | ∘ | ∘ | ∘ | ∘ | ∘ | ∘ |
| Bleeding | RN-3 | None | None | None | None | None | None | None | None | None |
| | RN-4 | None | None | None | None | None | None | None | None | None |
| | RN-5 | None | None | None | None | None | None | None | None | None |
| | RN-6 | None | None | None | None | None | None | None | None | None |
| | RN-7 | None | None | None | None | None | None | None | None | None |
| | RN-8 | None | None | None | None | None | None | None | None | None |

*∘; sufficient, Δ; slightly insufficient, x; insufficient
1 M = 1 month, 2 M = 2 months, 3 M = 3 months

TABLE 3
Examples of Patch formulations
RN-11, 14 and 17 containing Rivastigmine

| | Adhesive | | Lauroglycol | Plurol oleique | | |
|---|---|---|---|---|---|---|
| Patch | (17408) | Rivastigmine* | 90 | CC 497 | Labrasol | Thickness |
| RN-11 | 79 | 16 | 5 | | | 113 μm |
| RN-14 | 79 | 16 | | 5 | | 113 μm |
| RN-17 | 79 | 16 | | | 5 | 113 μm |

FIG. 2 shows the stability of drug content of the above formulations in different conditions.

TABLE 4
Examples of Patch formulations RN-18,
RN-19 and RN-20 containing Rivastigmine

| | Adhesive | | Lauroglycol | | Drug loading | Drug loading |
|---|---|---|---|---|---|---|
| Patch | (17408) | Rivastigmine* | 90 | Thickness | (mg/cm²) | ratio |
| RN-18 | 81 | 14 | 5 | 90 μm | 1.26 | 70 |
| RN-19 | 83 | 12 | 5 | | 1.08 | 60 |
| RN-20 | 85 | 10 | 5 | | 0.90 | 50 |

FIG. 3 shows the stability of drug content of the above formulations in different conditions.

TABLE 5
Stability Study of patch formulations RN-18, RN-19 and RN-20

| | | | 60° C. | | 40° C. |
|---|---|---|---|---|---|
| | Patch | Initial | 2 w | 1 M | 1 M |
| Crystallization | RN-18 | None | None | None | None |
| | RN-19 | None | None | None | None |
| | RN-20 | None | None | None | None |
| Adhesion* | RN-18 | ◯ | ◯ | ◯ | ◯ |
| | RN-19 | ◯ | ◯ | ◯ | ◯ |
| | RN-20 | ◯ | ◯ | ◯ | ◯ |
| Bleeding | RN-18 | None | None | None | None |
| | RN-19 | None | None | None | None |
| | RN-20 | None | None | None | None |

*◯; sufficient, Δ; slightly insufficient, X; insufficient
2 w = 2 weeks, 1 M = 1 month

As can be seen, all of the formulations according to the invention provided above good stability data for all three attributes, i.e., crystallization, adhesive and bleeding, at e.g., 60° C. and 40° C. for 1 month. There were no crystals were present in the patch as well as the absence of bleeding. Additionally, the above formulations showed sufficient adhesion and stayed on skin for at least 24 hours. Additionally, FIGS. 1, 2, 3 and 5 provided good stability data with respect to drug content in the above formulations.

TABLE 6
In vitro human skin permeation

| | Adhesive | | Lauroglycol | | Drug loading | Drug loading |
|---|---|---|---|---|---|---|
| Patch | (17408) | Rivastigmine | 90 | Thickness | (mg/cm²) | ratio |
| RN-18 | 81 | 14 | 5 | 90 μm | 1.26 | 70 |

| No. | Patch | Lot No. | Exp date |
|---|---|---|---|
| 3 | Exelon patch Lot 1 | 1942A | November 2013 |
| 4 | Exelon patch Lot 2 | 358210 | February 2014 |

Table 6 shows that RN-18 used only 14% of drug compared to Exelon, which is known to use 30%. Additionally, RN-18 still showed comparable skin permeation rate to that of Exelon's patch. The acrylic-hydrocarbon hybrid polymer according to the invention provided significantly high skin permeation rate compared to other adhesive, such as Exelon's acrylate. FIG. 4 demonstrates the comparative result of Table 6 (In vitro human skin permeation using Franz cell Mean±SE (n=4))

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

We claim:

1. A transdermal drug delivery system comprising a drug-containing matrix layer comprising rivastigmine or its pharmaceutically acceptable salt and an acrylic-hydrocarbon hybrid polymer.

2. The transdermal drug delivery system according to claim 1, further comprising a backing layer providing support for the pharmaceutical composition, an adhesive layer for contacting and fixing the pharmaceutical composition to the backing layer; and a release liner releasably contacting said adhesive.

3. The transdermal drug delivery system according to claim 1, wherein the acrylic-hydrocarbon hybrid polymer is an acrylic polymer comprising a C₄₋₁₈ alkyl acrylate monomer grafted within a hydrocarbon macromer having a glass transition temperature of not more than −30° C.

4. The transdermal drug delivery system according to claim 1, wherein rivastigmine or its pharmaceutically acceptable salt is present in an amount ranging from about 5% to about 40%, about 7% to about 30%, or about 10% to about 20% by weight on the total weight of the drug-containing matrix layer.

5. The transdermal drug delivery system according to claim 1, wherein the acrylic-hydrocarbon hybrid polymer is present in an amount ranging from about 60% to about 95%, about 70% to about 90%, or about 75% to about 85% by weight based on the total weight of the drug-containing matrix layer.

6. The transdermal drug delivery system according to claim 1 further comprising one or more absorption enhancers.

7. The transdermal drug delivery system according to claim 6, wherein the absorption enhancers is present in an amount ranging from about 1% to about 20%, or about 5% to about 15, by weight based on the total weight of the drug-containing matrix layer.

8. The transdermal drug delivery system according to claim 6, wherein the absorption enhancers are selected from the group consisting of terpenes, surfactants, polyoxyethylene alkyl ethers, fatty alcohols, sugar esters, glycerols, alkyl 2-ethyl hexanates and diethoxyethyl succinates.

9. The transdermal drug delivery system according to claim 6, wherein the absorption enhancers are selected from the group consisting of polyethylene glycol palm kernel glyceride, polyoxyethylene lauryl ether, polyglyceryl-3-oleate, lauryl alcohol and oleyl alcohol.

10. The transdermal drug delivery system according to claim 8, wherein the terpenes can be cineole or limonene.

11. The transdermal drug delivery system according to claim 8, wherein the surfactants are selected from the group consisting of isopropyl myristate, isopropyl palmitate, 2-(2-ethoxyethoxy) ethanol, oleic acid oleyl ester, caprylocaproyl macrogolglyceride, oleoyl macrogolglyceride, diisopropyl dirrerate, diisopropyl adipate, hexyl laurate, polysorbate, sorbitan oleate.

12. The transdermal drug delivery system according to claim 8, wherein the polyoxyethylene alkyl ethers are selected from the group consisting of polyethylene glycol palm kernel glyceride, 2-ethyl hexyl hydroxystearate, polyoxyethylene lauryl ether, and polyoxyethylene cetyl ether.

13. The transdermal drug delivery system according to claim 8, wherein the fatty alcohols are selected from the group consisting of polyglyceryl-3 oleate, polyethylene glycol almond glyceride, lauryl alcohol and oleyl alcohol.

14. The transdermal drug delivery system according to claim 8, wherein the sugar esters are selected from the group consisting of sucrose stearate, sucrose palmitate, sucrose laurate, sucrose behenate, sucrose oleate and sucrose erucate.

15. The transdermal drug delivery system according to claim 8, wherein the alkyl 2-ethyl hexanates are selected from the group consisting of 2-ethylhexanonate, cetyl 2-ethylhexanonate and stearyl 2-ethylhexanonate.

16. The transdermal drug delivery system of claim 1, which is in the form of a patch and the size of the patch ranges of from about 2.5 cm² to about 20 cm², from about 3.5 cm² to about 10.5 cm², from about 5 cm² to about 15 cm², about 5 cm², about 10 cm², or about 15 cm².

17. The transdermal delivery system according to claim 1, wherein the acrylic-hydrocarbon hybrid polymer is selected from the group consisting of 87-502A, 87-502B, 87-503A, 87-504A, 87-504B, and mixtures thereof.
